(19) United States
(12) Patent Application Publication
(10) Pub. No.: US 2017/0115215 A1
(43) Pub. Date: Apr. 27, 2017

(54) Sensor for detecting remotely located reflective material

(71) Applicants: Jeffrey Scott ADLER, Beaconsfield (CA); Harold Russell BAIRD, Marietta, GA (US)

(72) Inventors: Jeffrey Scott ADLER, Beaconsfield (CA); Harold Russell BAIRD, Marietta, GA (US)

(21) Appl. No.: 14/756,879

(22) Filed: Oct. 26, 2015

(51) Int. Cl. G01N 21/55; G01N 33/18

(52) U.S. Cl. CPC G01N 21/55; G01N 33/18; G01N 2201/062; G01N 2201/061; G01N 2033/1873

(57) ABSTRACT

A remote reflective materials sensor for detecting remotely located reflective material. The remote reflective materials sensor includes a transparent window with two window surfaces, an amount of reflective material that is remotely located away from one window surface. An operating parameters sensor located adjacent to the transparent window, a radiation detector located away from the other window surface; and two spaced apart radiation emitters located on either side of the radiation detector, and away from the second window surface. Each radiation emitter is configured to emit radiation along one axis through the transparent window towards the reflective material and towards a common focal point. The radiation detector is located to receive reflected radiation from the reflective material along another axis. The first axis of the radiation emitters is angled towards the other axis of the reflected radiation.

### TECHNICAL FIELD

The present relates to material sensors, and more particularly to a sensor for detecting remotely located reflective materials.

### BACKGROUND

Precipitation sensors have been developed to determine the presence of water in its vapor, liquid and solid forms, but usually the sensor is immersed in the material. Non-immersed sensing is a significant challenge. One example of a non-immersed sensor is the Bosch vehicle windshield rain sensor (Optical Sensor U.S. Pat. No. 6,376,824 by Michenfelder et al) used to operate windshield wipers. This sensor depends on the change in refraction of a reflected light beam against glass when water is on the outer glass surface. However, it has poor sensitivity for snow, unless the glass can be heated enough to melt the snow next to the glass. Moreover, it is unable to detect snow above the glass surface. The ability to remotely detect reflective material at a distance away can be very valuable for numerous applications that encounter reflective material (such as winter precipitation or ice formed from super cooled water droplets) wherein there may be adverse effects encountered unless otherwise detected. Examples of which include detection of mid-air ice or super cooled water in the aerospace industry which would certainly welcome such an invention since ice detection has been a challenge for many years. An example of a sensor determining the presence of a material above a surface using reflection techniques is U.S. Pat. No. 8,741,513 B2 Rain Sensor by Han. Disadvantageously, Han's sensor requires that the material be on the surface far side and be translucent so that the radiation can reflect off the material back surface through the translucent material in order to be sensed. Thus it will not detect clear ice which has a poor backside reflection. It is also susceptible to false signals such as sunlight, oncoming headlights, or street lamps shining through and mimicking a reflective signal. Han teaches that light sources and light receivers mounted on a plane inclined at an angle to the window prevent sources light from being reflected off the window surface back to light receivers. But drawing a complete set of light vectors on Han's art yields paths where source light is obviously reflected off the window surface to the light receivers, significantly reducing sensitivity to raindrop reflection signals. This loss of sensitivity is especially problematic, given that the majority of source light will pass through the raindrop rather than be reflected, and external light can also pass easily through the raindrop causing false signals to the light receivers. Han teaches the use of infrared light sources and receivers. Han's sensor specifically detects reflection from the far side of raindrops on a window surface. Han teaches a lattice pattern of light sources and receivers, suitable for the short distances in a windshield wiper control.

Another example U.S. Pat. No. 8,873,062 by Adler & Baird is described as a sensor for sensing reflective material specifically on the transparent window surface of the sensor. The surface sensor is not able to detect reflective material remotely or at a distance. While the sensor does work well in terms of detection on the transparent surface, it does not have the capabilities to effectively detect away from the surface as provided for by the application herein that provides for the use of a radiation detector focusing lens and varied radiation emitters of differing intensities and/or radiation frequencies allowing for operational flexibility in numerous light conditions, to detect reflective materials at a distance away.

Another example is U.S. Pat. No. 7,285,771 B2 Optical Sensor by Walker. Walker teaches the use of a single emitter and several detectors to determine paper position in a printer. While this sensor works well in the enclosed environment of a printer, with one emitter it does not have the flexibility to accommodate various light conditions found in other applications. Walker teaches using an infrared emitter and an enclosed housing to minimize the effect of stray light. This restricts the use to reflective material located within enclosed housing environments. Walker teaches an open air path for the radiated and reflected light. This is suitable for the benign environment in an enclosed housing in a printer.

Thus there is a need for a sensor useful in detecting remotely located reflective material

### BRIEF SUMMARY

We have discovered through design, trial and error a remote reflective materials sensor that uses a reflective rather than refractive technique, and as such is very well suited to determining the presence of reflective materials located at a distance remote from a surface. A radiation source such as a Light Emitting Diode (LED) is oriented to radiate at a distance through a transparent material such as glass. In one example, the transparent material is a window. When a reflective material such as snow or rime ice is within the sensor's field of view, a radiation detector such as but not limited to a phototransistor, photo diode or light dependent resister adjacent to the radiation source, detects the radiation reflection.

Our discovery uses two radiation emitters angled to a common focal point, this focal point also being common with the radiation detector. The radiation emitters may be of differing intensities or radiation frequencies, allowing for operational flexibility in a variety of ambient light conditions and detecting a variety of reflective materials. A single radiation emitter may also be used in applications not requiring the radiation intensity or radiation frequency features of two emitters. Our discovery uses a mount, which also functions as a light baffle to prevent sensitivity degrading false reflections. Our device uses a variety of emitter and detector types, which can be modified depending on to the reflective material to be detected. Furthermore, our device uses detector path focusing lenses in the detector array embodiment that can extend the sensor range without increasing radiation emitter power. Moreover, our device provides for a temperature sensor adjacent to the transparent window to distinguish between liquid and frozen reflective materials expected in a given application. While direct sensing of the transparent window temperature provides the best accuracy, alternate methods may be used. An example is a temperature sensor located elsewhere in the housing. Another example that may be used in a turbine engine application combines remote air temperature sensing from an aircraft fuselage mounted probe with algorithmic processing of engine parameters such as RPM to deduce engine temperature at the transparent window location. For example, engine air compression ratio at any compressor stage as a function of airspeed and engine RPM is determined in prototype development, or the engine may be instrumented with a pressure sensor. A thermodynamic equation can then be used to relate measured ambient air temperature to compressor stage temperature using compression ratio. Another example which may be used is an algorithmic only approach, whereby a combination of application operating parameters such as altitude and RPM can be used to deduce temperature at the transparent window position. Other methods may also be used, based on a person of ordinary skill in the art applying methods available in any given application.

We are unaware of any devices that currently detect reflective materials at a distance from the window. Our device uses one or more emitters aligned to a common target area outside the housing to produce a usable signal under varying ambient light conditions and reflective material locations outside the housing and away from it. We describe radiating through and receiving reflected light through a housing transparent window. This is suitable for a harsh environment such as detecting reflective material on a turbine engine compressor guide vane.

Furthermore, radiating through a transparent window allows the device to be used where the window isolates the sensor from harsh environments such as high or low temperatures or pressures, corrosive, toxic or flammable materials, and the like. The transparent window is also easier to clean than emitters and detectors. The transparent window material may also be selected to be transparent to the emitter and/or detector radiation wavelengths, but filter out undesirable ambient radiation.

Accordingly, there is provided a remote reflective materials sensor for detecting remotely located reflective material, the remote reflective materials sensor comprising:
a transparent window having first and second window surfaces, an amount of reflective material being remotely located away from the first window surface;
an operating parameters sensor located adjacent to the transparent window;
a radiation detector located away from the second window surface; and
first and second spaced apart radiation emitters located on either side of the radiation detector, and away from the second window surface, each radiation emitter being configured to emit radiation along a first axis through the transparent window towards the reflective material and towards a common focal point, the radiation detector being located to receive reflected radiation from the reflective material along a second axis, the first axis of the radiation emitters being angled towards the second axis of the reflected radiation.

In one example, the remote reflective materials sensor further includes a housing which houses a sensor mount, the radiation detector and the radiation emitters being mounted in the sensor mount. The sensor mount includes two spaced apart cavities aligned along the respective first axes in which the radiation emitters are located, and another cavity aligned along the second axis in which the radiation detector is located.

In one example, the operating parameters sensor is selected from the group consisting of: a temperature sensor, a pressure sensor, an airspeed sensor, an RPM sensor, and an altitude sensor.

In one example, the radiation emitter is a Light Emitting Diode (LED).

In one example, the radiation emitter is an electroluminescent surface.

In one example, the radiation emitter is a narrow beam high radiation emitter. The remote reflective materials sensor, according to claim 7, in which the narrow beam high radiation emitter is a laser, or a focused emitter, the focused emitter including a focused LED, a focused incandescent bulb, or a focused electric arc.

In one example, the radiation detector is a photo transistor, a photo diode or a light dependent resister located adjacent to the radiation emitter to detect reflected radiation.

In one example, the radiation detector is an array of detectors to detect spatially separated reflective material elements including individual snowflakes, ice crystals, or successive positions of one reflective object in the sensor field of view.

In one example, the first and second radiation emitters and the housing are configured so that radiation is emitted through the transparent window without causing false radiation reflection back to the radiation detector.

In one example, a controller is located in the housing and is connected to a variable resistor, the radiation detector, the radiation emitter and the operating parameters sensor.

In one example, a controller is located in the housing and is connected to a fixed resistor, the radiation detector, the radiation emitter and the operating parameters sensor.

In one example, the radiation detector is an integrated circuit having a phototransistor, a photo diode or a light dependent resister located adjacent to the radiation emitter so as to detect reflected radiation.

In one example, the reflective material is winter precipitation. The winter precipitation is snow, sleet, frost, ice or ice pellets.

In one example, the reflective material is non-winter precipitation. The non-winter precipitation is reflective liquids, dirt, particulate material suspended in liquids, super cooled water droplets, or ice, including clear and rime ice.

According to another aspect, there is provided a use of the remote reflective materials sensor to detect reflective material located remote from the transparent window and associated with: airplanes, helicopters, drones, unmanned air vehicles, spacecraft, blimps, hybrid air/ground/marine/space vehicles, trucks, cars, motor bikes, recreational vehicles, trains, boats; sidewalks, driveways, walkways, roads, roofs, greenhouses, atriums, windows, skylights; food services, food preparation and preservation, freezer glass doors, freezers and/or refrigerators, buildings or infrastructure projects, medical applications including storage of tissues and cells, or sterilizations; landscaping including grass and garden maintenance, or crops weather determination, agriculture, climate, and ecosystem preservation; or energy production applications including solar applications for building materials including decking, walls or shingles.

In one example, the transparent window is made from a material that is transparent to emitter and detector radiation, and filters ambient radiation.

Accordingly in another aspect, there is provided a remote reflective materials sensor for detecting remotely located reflective material, the remote reflective materials sensor comprising:
a transparent window having first and second window surfaces, an amount of reflective material being remotely located away from the first window surface;
a radiation detector located away from the second window surface; and
first and second spaced apart radiation emitters located on either side of the radiation detector, and away from the second window surface, each radiation emitter being configured to emit radiation along a first axis through the transparent window towards the reflective material and towards a common focal point, the radiation detector being located to receive reflected radiation from the reflective material along a second axis, the first axis of the radiation emitters being angled towards the second axis of the reflected radiation, the first and second radiation emitters being located so as to avoid interference with the reflected radiation.

Accordingly in another aspect, there is provided a remote reflective materials sensor for detecting remotely located reflective material, the remote reflective materials sensor comprising:
a transparent window having first and second window surfaces, an amount of reflective material being remotely located away from the first window surface;
a combination of an operating parameters sensor located away from the sensor and an algorithm to determine window temperature;
a radiation detector located away from the second window surface;
first and second spaced apart radiation emitters located on either side of the radiation detector, and away from the second window surface, each radiation emitter being configured to emit radiation along a first axis through the transparent window towards the reflective material and towards a common focal point, the radiation detector being located to receive reflected radiation from the reflective material along a second axis, the first axis of the radiation emitters being angled towards the second axis of the reflected radiation, the first and second radiation emitters being located so as to avoid interference with the reflected radiation.

In one example, the operating parameters sensor is selected from the list of a temperature sensor, a pressure sensor, an airspeed sensor, an RPM sensor, and an altitude sensor.

Accordingly in another aspect, there is provided a remote reflective materials sensor for detecting remotely located reflective material, the remote reflective materials sensor comprising:
a transparent window having first and second window surfaces, an amount of reflective material being remotely located away from the first window surface;
an operating parameters sensor located adjacent to the transparent window;
a radiation detector located away from the second window surface; and
a radiation emitter located adjacent the radiation detector, and away from the second window surface, the emitter being configured to emit radiation along a first axis through the transparent window towards the reflective material, the radiation detector being located to receive reflected radiation from the reflective material along a second axis, the first axis of the radiation emitter being angled towards the second axis of the reflected radiation.

In one example, the remote reflective materials sensor, includes two spaced apart radiation emitters located on either side of the radiation detector, and away from the second window surface, each radiation emitter being configured to emit radiation along a first axis through the transparent window towards the reflective material and towards a common focal point.

### BRIEF DESCRIPTION OF THE DRAWINGS

In order that the discovery may be readily understood, embodiments are illustrated by way of example in the accompanying drawings.

FIG. 1A illustrates top view of a remote reflective materials sensor;

FIG. 1B illustrates a side view of the remote reflective materials sensor showing radiation emitted and radiation reflected;

FIG. 2 illustrates the remote reflective materials sensor's field of view;

FIG. 3 is an alternate embodiment illustrating the radiation detector array;

FIG. 4 is diagrammatic representation of communication between remote reflective materials sensor components in one example of the sensor; and

FIG. 5 is diagrammatic representation of communication between remote reflective materials sensor components in an alternative example of the sensor.

Further details of the device and its advantages will be apparent from the detailed description included below.

### DETAILED DESCRIPTION

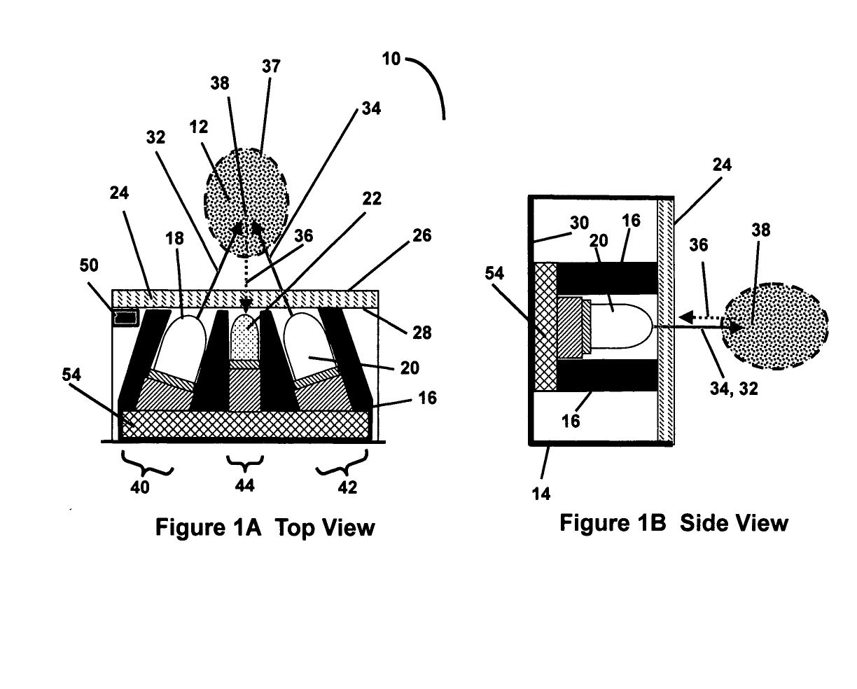

Referring to FIGS. 1A, and 1B, there is illustrated generally at 10 a remote reflective materials sensor for sensing reflective material 12 at a distance. In one example, the reflective material is winter precipitation such as, for example, snow, frost, ice or ice pellets. In another example, the reflective material is non-winter precipitation such as reflective liquids, dirt, super cooled water drops including clear and rime ice, or particulate material suspended in liquids. Broadly speaking, the remote reflective materials sensor 10 includes a housing 14, a sensor mount 16, two radiation emitters (radiation sources) 18, 20, a radiation detector 22 and a controller 54. The housing 14 has a transparent window 24 with first surface 26 and second surface 28, providing visibility of the reflective material 12 to the radiation emitters 18 and 20 and the radiation detector 22. The sensor mount 16 is located in the housing 14. The radiation emitters 18, 20 are mounted in the sensor mount 16. The radiation emitter 18, 20 each have a first axis 32, 34. Radiation is emitted from the radiation emitters 18, 20 along their respective axes 32, 34 towards and through the transparent window 24 until it contacts the distanced reflective material 12 away from the first surface 26 of the transparent window 24. The radiation detector 22 is mounted in the sensor mount 16 and adjacent and between the radiation emitters 18, 20. The radiation detector 22 and the radiation emitters 18, 20 are located away from the second surface 28 of the transparent window 24. The radiation detector 22 is located to receive the radiation that is reflected back from the distanced reflective material 12 along a second axis 36. The first axes 32, 34 of the radiation emitters 18, 20 are both angled towards the second axis 36. The two radiation emitters 18, 20 emit radiation towards a common focal point 38 on the distanced reflective material 12. The sensor mount 16 prevents radiation emitter 18, 20 radiation from being mirror reflected to the radiation detector 22 by the transparent window 24. The radiation detector 22 is directed to the radiation emitter common focal point 38 on the distanced reflective material 12.

The temperature sensor only detects temperature. It does not detect barometric pressure or other parameters. It should be noted that the remote reflective materials sensor 10 will function without a temperature sensor. Without temperature, the reflective material sensor 10 assumes that any reflection is the reflective material ice of interest, and not dirt. With the temperature sensor, any reflection measured above freezing temperature can be assumed to be a foreign substance, allowing the implementation to trigger a maintenance operation by a technician.

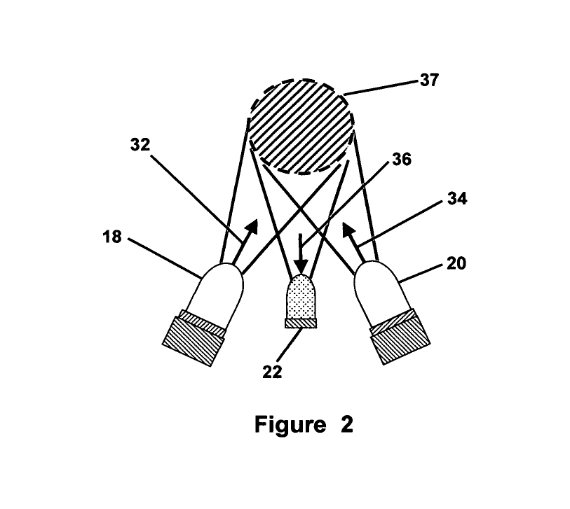

Referring briefly to FIG. 2, radiation emitters 18 and 20 and radiation detector 22 have overlapping fields of useful radiation and detection to sense precipitation over area 37.

One skilled in the art will recognize that the reflective material sensor range can be extended by using narrow beam high radiation emitters such as, for example, lasers, focused LEDs, focused incandescent bulbs, or focused electric arcs.

Still referring to FIGS. 1A, and 1B, the sensor mount 16 includes two spaced apart cavities 40, 42 which are both aligned along their respective first axes 32, 34 in which the radiation emitters 18, 20 are located. Another cavity 44 is aligned along the second axis 36 in which the radiation detector 22 is located.

Still referring to FIG. 1A, a temperature sensor 50 is located adjacent to the transparent window 24 out of the radiation detector's 22 field of view, which will not cause a false reflection to the radiation detector.

By way of example each of the radiation emitters is a Light Emitting Diode (LED).

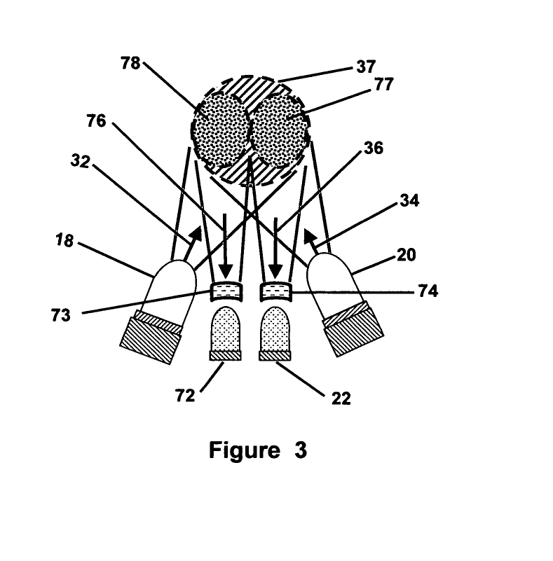

Referring now to FIG. 3, an alternate embodiment is illustrated which is capable of detecting more than one reflective material element at a distance, and is therefore capable of detecting a moving or increasing or decreasing size of reflective material over time. Radiation detector 22 and at least one radiation detector 72 utilize where necessary focusing device 74 and where necessary at least one focusing device 73 to sense reflective material element 77 and at least one reflective material element 78.

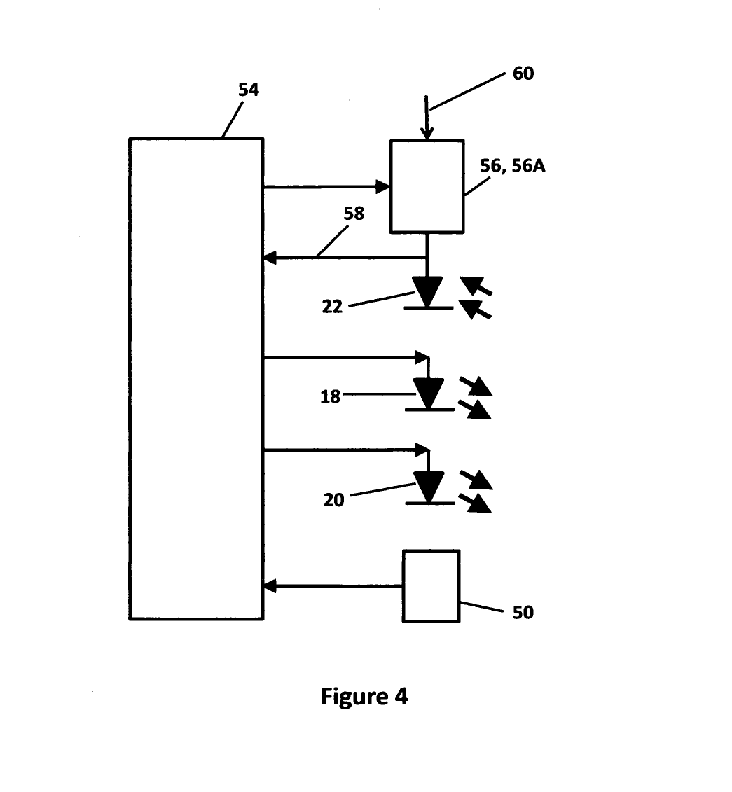

Referring now to FIG. 1A, FIG. 1B and FIG. 4, a controller 54, which is typically a microprocessor or equivalent device, communicates with an included variable resister 56 or fixed resister 56A, the radiation detector 22, the radiation emitters 18, 20 and the temperature sensor 50 to achieve the reflective material sensing function. The controller 54 may be located within the housing 14, or in another suitable housing. One skilled in the art will understand that other devices and circuitry such as cabling, voltage supply, ground, signal buffering, user communication, controller programming, additional radiation detectors and associated variable or fixed resisters, and the like may also be integrated into the sensing function.

Referring now to FIG. 4, the radiation detector 22 operates as an electrical current valve, which permits higher current flow at higher radiation levels. A reference voltage 60 is passed through the variable resister 56 and then the radiation detector to produce a radiation signal 58. As radiation increases, the current flow through the radiation detector 22 increases, causing an increased voltage drop across the variable resister 56. To allow for a wide range of radiation, the controller 54 modifies the value of the variable resister 56 to produce a usable signal. For installations where the ambient radiation range is small, an inexpensive fixed resister 56A may be used, thereby eliminating the need for the controller 54 to modify the resister 56A value. Alternatively, more than one copy of a fixed but different value resister 56A and radiation detector 22 may be used to broaden the sensed radiation range to provide maximum sensor detection of reflective material at a distance.

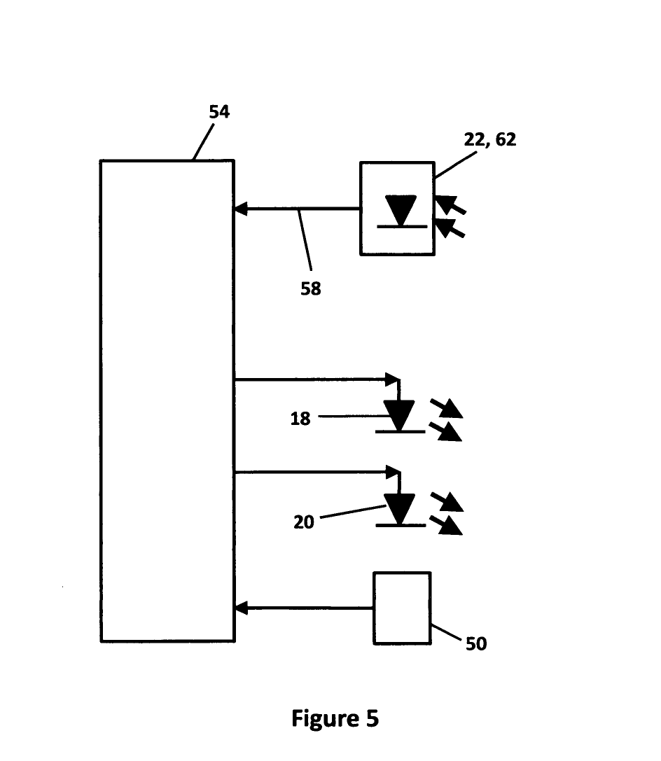

Referring now to FIGS. 1 and 5, the radiation detector 22 is an integrated circuit 62, which includes a radiation detector such as a photo diode, photo transistor or light dependent resister and a means to autonomously convert the radiation detector 22 output to the controller 54 compatible input such as frequency pulses.

Still referring to FIG. 4 or 5, the controller 54 activates one or both of the radiation emitters 18, 20 when required to achieve the sense function. To assist in distinguishing between frozen and liquid reflective material, the controller 54 communicates with the temperature sensor 50 to determine whether frozen reflective material is possible.

The reflective material sensor 10 functions in a wide range of ambient radiations, from direct sunlight to nighttime. It can remotely sense reflective material on or at a distance away from for example, greenhouses, atriums, windows, freezer glass doors, skylights; on airplanes, drones, helicopters, spacecraft, aircraft, hybrid air/space/water/land vehicle components, and motorized transportation including trucks, cars, motor bikes, recreational vehicles, trains, boats and the like; food services, freezers/fridges, buildings, photovoltaic solar (conventional panels and non conventional solar applications), trough reflectors; for landscaping such as grass and garden maintenance, crops; or for weather determination, climate, ecosystem preservation; or for medical applications and storage of tissues and cells, sterilizations; or for food preparation and preservation, and the like. When operated in non-winter conditions, the remote reflective materials sensor 10 may also detect dirt on these types of surfaces to support cleaning operations. It can also detect ice crystal accretion in the atmosphere, which may not necessarily be associated with winter conditions. The remote reflective materials sensor 10 can also sense winter precipitation when installed in sidewalks, driveways, walkways, roads, roofs, infrastructure projects and the like. The remote reflective materials sensor 10 can be used in solar applications for building materials such as decking, walls and shingles.

While the remote reflective materials sensor 10 can be used to sense winter precipitation, it is easily applied to sensing other reflective materials such as, for example, liquids, precipitates, contamination, some gases, suspended solids, and the like, and as such can be applied to manufacturing and distribution processes for food, chemicals, fuels, and the like.

### Operation

Referring now to FIGS. 1A, 1B and 4, operation of the remote reflective materials sensor 10 will be described. Distanced reflective material 12 is detected by determining the change in the radiation signal 58 when the radiation emitters 18, 20 are “off” then “on”. Firstly, the controller 54 determines if reflective material 12 is possible by communicating with the temperature sensor 50 and any available external sources of data. If reflective material 12 is possible, then the controller 54 determines a reference ambient radiation signal 58 by first not switching on the radiation emitters 18, 20, then modifying the variable resister 56 until the radiation signal 58 is approximately 90% of the reference voltage 60. The controller 54 determines the reference ambient radiation by comparing the resultant variable resister 56 resistance with internally stored data. If the fixed resister 56A is used, the controller 54 determines reference ambient radiation by comparing the radiation signal 58 with internally stored data.

Referring now to FIG. 5, an alternative operation of the remote reflective materials sensor 10 will now be described. Distanced reflective material 12 is detected by determining the change in the radiation signal 58 when the radiation emitters 18, 20 are “off” then “on”. Firstly, the controller 54 determines if reflective material 12 is possible by communicating with the temperature sensor 50 and any available external sources of data. If reflective material 12 is possible, then the controller 54 determines a reference ambient radiation signal by first not switching on the radiation emitters 18, 20 then communicating with the radiation detector 62.

Referring now to FIGS. 4 and 5, the controller 54 then turns on one or both of the radiation emitters 18, 20 depending on the ambient radiation. At high ambient radiation, both radiation emitters 18, 20 may be required to obtain an adequate change in the radiation signal 58. The controller 54 then determines that reflective material 12 is present at a distance if the radiation signal 58 value has changed from the reference ambient radiation signal value by more than the combined effect of impurities in the transparent window 24 and expected dirt on the transparent window 24. The controller 54 may also determine the type of distanced reflective material 12 based on the combination of the temperature sensor 50 and the radiation signal 58 change, and the type of radiation emitter(s) and radiation detector(s) used.

In some applications such as low ambient light or reflective material easily detected at one radiation frequency, the dual emitter operations can be readily simplified for single emitter applications by anyone skilled in the art.

It should be noted that in the Figures, the area shown as the reflective material 12 is the sensor illumination or the detection coverage area. The reflected radiation signal will vary from a low value with no reflective material in the detection coverage area to a high value with highly reflective material covering the entire detection coverage area.

### OTHER EMBODIMENTS

From the foregoing description, it will be apparent to one of ordinary skill in the art that variations and modifications may be made to the embodiments described herein to adapt it to various usages and conditions.

We claim:

1. A remote reflective materials sensor for detecting remotely located reflective material, the remote reflective materials sensor comprising:
a transparent window having first and second window surfaces, an amount of reflective material being remotely located away from the first window surface;
an operating parameters sensor located adjacent to the transparent window;
a radiation detector located away from the second window surface; and
first and second spaced apart radiation emitters located on either side of the radiation detector, and away from the second window surface, each radiation emitter being configured to emit radiation along a first axis through the transparent window towards the reflective material and towards a common focal point, the radiation detector being located to receive reflected radiation from the reflective material along a second axis, the first axis of the radiation emitters being angled towards the second axis of the reflected radiation.

2. The remote reflective materials sensor, according to claim 1, further includes a housing which houses a sensor mount, the radiation detector and the radiation emitters being mounted in the sensor mount.

3. The remote reflective materials sensor, according to claim 2, in which the sensor mount includes two spaced apart cavities aligned along the respective first axes in which the radiation emitters are located, and another cavity aligned along the second axis in which the radiation detector is located.

4. The remote reflective materials sensor, according to claim 1, in which the operating parameters sensor is selected from the group consisting of: a temperature sensor, a pressure sensor, an airspeed sensor, an RPM sensor, and an altitude sensor.

5. The remote reflective materials sensor, according to claim 1, in which the radiation emitter is a Light Emitting Diode (LED).

6. The remote reflective materials sensor, according to claim 1, in which the radiation emitter is an electroluminescent surface.

7. The remote reflective materials sensor, according to claim 1, in which the radiation emitter is a narrow beam high radiation emitter.

8. The remote reflective materials sensor, according to claim 7, in which the narrow beam high radiation emitter is a laser, or a focused emitter, the focused emitter including a focused LED, a focused incandescent bulb, or a focused electric arc.

9. The remote reflective materials sensor, according to claim 1, in which the radiation detector is a photo transistor, a photo diode or a light dependent resister located adjacent to the radiation emitter to detect reflected radiation.

10. The remote reflective materials sensor, according to claim 1, in which the radiation detector is an array of detectors to detect spatially separated reflective material elements including individual snowflakes, ice crystals, or successive positions of one reflective object in the sensor field of view.

11. The remote reflective materials sensor, according to claim 1, in which the first and second radiation emitters and the housing are configured so that radiation is emitted through the transparent window without causing false radiation reflection back to the radiation detector.

12. The remote reflective materials sensor, according to claim 2, in which in which a controller is located in the housing and is connected to a variable resistor, the radiation detector, the radiation emitter and the operating parameters sensor.

13. The remote reflective materials sensor, according to claim 2, in which a controller is located in the housing and is connected to a fixed resistor, the radiation detector, the radiation emitter and the operating parameters sensor.

14. The remote reflective materials sensor, according to claim 1, in which the radiation detector is an integrated circuit having a phototransistor, a photo diode or a light dependent resister located adjacent to the radiation emitter so as to detect reflected radiation.

15. The remote reflective materials sensor, according to claim 1, in which the reflective material is winter precipitation.

16. The remote reflective materials sensor, according to claim 15, in which the winter precipitation is snow, sleet, frost, ice or ice pellets.

17. The remote reflective materials sensor, according to claim 1, in which the reflective material is non-winter precipitation.

18. The remote reflective materials sensor, according to claim 17, in which the non-winter precipitation is reflective liquids, dirt, particulate material suspended in liquids, super cooled water droplets, or ice, including clear and rime ice.

19. Use of the remote reflective materials sensor, according to claim 1, to detect reflective material located remote from the transparent window and associated with: airplanes, helicopters, drones, unmanned air vehicles, spacecraft, blimps, hybrid air/ground/marine/space vehicles, trucks, cars, motor bikes, recreational vehicles, trains, boats; sidewalks, driveways, walkways, roads, roofs, greenhouses, atriums, windows, skylights; food services, food preparation and preservation, freezer glass doors, freezers and/or refrigerators, buildings or infrastructure projects, medical applications including storage of tissues and cells, or sterilizations; landscaping including grass and garden maintenance, or crops weather determination, agriculture, climate, and ecosystem preservation; or energy production applications including solar applications for building materials including decking, walls or shingles.

20. The remote reflective materials sensor, according to claim 1, in which the transparent window is made from a material that is transparent to emitter and detector radiation, and filters ambient radiation.

21. A remote reflective materials sensor for detecting remotely located reflective material, the remote reflective materials sensor comprising:
a transparent window having first and second window surfaces, an amount of reflective material being remotely located away from the first window surface;
a radiation detector located away from the second window surface; and
first and second spaced apart radiation emitters located on either side of the radiation detector, and away from the second window surface, each radiation emitter being configured to emit radiation along a first axis through the transparent window towards the reflective material and towards a common focal point, the radiation detector being located to receive reflected radiation from the reflective material along a second axis, the first axis of the radiation emitters being angled towards the second axis of the reflected radiation, the first and second radiation emitters being located so as to avoid interference with the reflected radiation.

22. A remote reflective materials sensor for detecting remotely located reflective material, the remote reflective materials sensor comprising:
a transparent window having first and second window surfaces, an amount of reflective material being remotely located away from the first window surface;
a combination of an operating parameters sensor located away from the remote reflective materials sensor and an algorithm to determine window temperature;
a radiation detector located away from the second window surface;
first and second spaced apart radiation emitters located on either side of the radiation detector, and away from the second window surface, each radiation emitter being configured to emit radiation along a first axis through the transparent window towards the reflective material and towards a common focal point, the radiation detector being located to receive reflected radiation from the reflective material along a second axis, the first axis of the radiation emitters being angled towards the second axis of the reflected radiation, the first and second radiation emitters being located so as to avoid interference with the reflected radiation.

23. The remote reflective materials sensor, according to claim 22, in which the operating parameters sensor is selected from the list of a temperature sensor, a pressure sensor, an airspeed sensor, an RPM sensor, and an altitude sensor.

24. A remote reflective materials sensor for detecting remotely located reflective material, the remote reflective materials sensor comprising:
a transparent window having first and second window surfaces, an amount of reflective material being remotely located away from the first window surface;
an operating parameters sensor located adjacent to the transparent window;
a radiation detector located away from the second window surface; and
a radiation emitter located adjacent the radiation detector, and away from the second window surface, the emitter being configured to emit radiation along a first axis through the transparent window towards the reflective material, the radiation detector being located to receive reflected radiation from the reflective material along a second axis, the first axis of the radiation emitter being angled towards the second axis of the reflected radiation.

25. The remote reflective materials sensor, according to claim 24, includes two spaced apart radiation emitters located on either side of the radiation detector, and away from the second window surface, each radiation emitter being configured to emit radiation along a first axis through the transparent window towards the reflective material and towards a common focal point.
